(19) United States
(12) Patent Application Publication
(10) Pub. No.: US 2015/0359627 A1
(43) Pub. Date: Dec. 17, 2015

(54) System and Method for Heart Valve Anchoring

(71) Applicant: MICRO INTERVENTIONAL DEVICES, INC., Newtown, PA (US)

(72) Inventor: Michael P. Whitman, Newtown, PA (US)

(21) Appl. No.: 14/737,408

(22) Filed: Jun. 11, 2015

Related U.S. Application Data
(60) Provisional application No. 62/010,680, filed on Jun. 11, 2014.

(51) Int. Cl. A61F 2/24

(52) U.S. Cl. CPC A61F 2/243; A61F 2/2409; A61F 2/2412; A61F 2/2427

(57) ABSTRACT

A system and method for percutaneous heart valve replacement includes implanting a heart valve replacement prosthetic into tissue and driving anchors into the heart valve replacement to affix the prosthetic to the tissue.

### CROSS-REFERENCE TO RELATED APPLICATIONS

This application claims the benefit of, and incorporates by reference thereto in its entirety, U.S. Provisional Patent Application Ser. No. 62/010,680, filed on Jun. 11, 2014.

Further, each of the following is hereby incorporated by reference thereto in its entirety: U.S. patent application Ser. No. 14/321,476, filed Jul. 1, 2014, U.S. patent application Ser. No. 14/301,106, filed Jun. 10, 2014, U.S. patent application Ser. No. 13/843,930, filed Mar. 15, 2013, PCT Application No. PCT/US14/30868, filed Mar. 17, 2014, U.S. patent application Ser. No. 13/010,769, filed Jan. 20, 2011, U.S. Provisional Patent Application Ser. No. 61/296,868, filed on Jan. 20, 2010, U.S. patent application Ser. No. 13/010,766, filed on Jan. 20, 2011, U.S. patent application Ser. No. 13/010,777, filed on Jan. 20, 2011, and U.S. patent application Ser. No. 13/010,774, filed on Jan. 20, 2011.

### FIELD OF THE INVENTION

The present invention relates to a system and method of percutaneous heart valve replacement, including anchoring the heart valve replacement into tissue.

### BACKGROUND INFORMATION

Heart valve replacements have been developed to counter heart valve failure, either from heart valve regurgitation (i.e., the failure of the heart valve to properly close), or from heart valve stenosis (i.e., the failure of the heart valve to properly open). Though early efforts at heart valve repair and replacement included open surgery, more recent developments have included percutaneous surgical applications.

Percutaneous heart valve repair, however, has shown certain disadvantages. For example, percutaneous repair involves modified surgical techniques, which can limit the benefits of the procedure. Annular rings may lack effectiveness, and include risks of erosion, perforation, and coronary artery thrombosis. Edge-to-edge repair can be technically demanding, and may lack long term durability. Depending upon the particular valve failure, combinations of different repair techniques may be necessary, further complicating the procedure and limiting its effectiveness.

In contrast, heart valve replacement has provided certain advantages, limiting the risks associated with heart valve repair, and applying to a broader scope of patients. Open surgery solutions for heart valve replacement, however, carry significant risks to the patient. Therefore, a less invasive, percutaneous heart valve replacement is needed.

Existing percutaneous solutions include U.S. Pat. No. 7,621,948, describing a percutaneously inserted heart valve prosthesis, which can be folded inside a catheter for delivery to the implant location. Another percutaneous solution is available from CardiAQ Valve Technologies, Inc., described in U.S. Patent Application Publication No. 2013/0144378. Other percutaneous prosthetic valves include Neovasc Tiara, Valtech Cardiovalve, ValveXchange, Lutter Valve, and valves from Medtronic, Inc. and Edwards Lifesciences Corporation.

In providing a percutaneous heart valve replacement, challenges include providing an implant that may be folded into a catheter for delivery, and can emerge from the catheter to fit properly into the implant site and serve its function as a valve. The implant valve must therefore be small enough to be folded into the catheter, but must be large enough, upon implantation, to provide the functions of the valve, without being so large as to obstruct ventricular flow.

Moreover, fixing the heart valve implant to the implant site may be challenging, as the implant site may form an irregular shape, may lack calcium to secure the valve, or may cause difficulty in fixing the implant valve with the proper orientation.

There is a need for a percutaneous heart valve solution to sufficiently and effectively address these challenges.

### SUMMARY

In accordance with example embodiments of the present invention, a device for delivering, implanting, and fixing to tissue a heart valve replacement prosthetic is provided. The device may include a ring, which may be pliable enough to be folded into a small space, such as the cavity of a catheter. The ring may be elastic, so as to automatically expand upon its release from its folded position in a catheter. Where an implant site is irregularly shaped, the elasticity of the ring permits the ring to form to the shape of the implant site. The device may further include one or more leaflets connected to the ring, effective to block fluid flow in a first fluid flow direction and to permit fluid flow in a second fluid flow direction.

The system of the present invention may include an applicator, including an applicator shaft passing through the ring, and terminating at its distal end in one or more spring arms. The spring arms may connect the distal end of the applicator shaft to the ring, such that the spring arms exert a spring force against the ring, pushing the ring radially outward. Each spring arm is separately connected to the ring, so that, in the case of a plurality of spring arms, each spring arm may each respond individually to an irregularly shaped implant site, allowing the ring to form to the shape of the implant site, and provide a better seat for the prosthetic heart valve.

The prosthetic may be delivered to the implant site percutaneously, for example, by a catheter, into which the prosthetic may be folded. Once delivered to the implant site, the prosthetic may be pushed through a distal end of the catheter, such that the ring is allowed to expand. The prosthetic may then be implanted in the implant site, with the elasticity of the ring and the independent spring arms permitting the prosthetic to form to any irregular shape of the implant site.

Once implanted, in an example embodiment of the present invention, a driver may be used to drive anchors through the ring, and into the tissue of the implant site, fixing the prosthetic to the tissue of the implant site. The driver may be configured to drive one or more anchors into one or more positions about the ring, and may index the driving of anchors in line with each spring arm. Once the prosthetic has been fixed to the implant site, the applicator may be withdrawn, leaving the prosthetic fixed in place. The prosthetic may have a smaller width or diameter than the width or diameter of the implant site, such that, once the anchors have been driven into the surrounding tissue of the implant site, the tissue of the implant site may be drawn toward the smaller prosthetic, to meet the exterior shape of the prosthetic.

In this manner, the prosthetic valve may be securely fixed to the implant site, to more sufficiently and effectively improve valve function. The prosthetic may take a variety of shapes. The shape of the prosthetic to be used for a particular application may be selected based on the particular geometric needs of the particular application.

In accordance with example embodiments of the present invention, a surgical device includes an applicator having an applicator shaft passing through the ring and terminating at a distal end having one or more spring arms connecting the distal end of the applicator shaft to the ring, such that a proximal force applied to the applicator shaft is transferred to the one or more spring arms and to the pliable ring, and a driver having a guide situated annularly about the applicator shaft, and at least one firing arm including at least one anchor outlet, the driver configured to slide between a proximal position and a distal position along the applicator shaft, wherein the at least one firing arm is in hinged communication with the driver, such that in the proximal position of the driver the at least one anchor outlet is situated in parallel with the applicator shaft, and in the distal position of the driver the at least one anchor outlet is directed toward the pliable ring, and the driver is configured to exert a driving force on at least one anchor to drive the anchor into the pliable ring.

The prosthetic valve may be delivered to an implant site by a catheter, the pliable ring being folded for insertion into the catheter and expandable once pushed from a distal end of the catheter. The prosthetic valve may be implanted at an implant site, and the driver may be configured to drive the anchor at least partially through the ring and into tissue surrounding the implant site.

The anchor may include a distal end tapered to a distal tip configured to pierce tissue, at least one barb extending proximally and radially outwardly from the distal end to a free end including a radially exterior surface and a radially interior surface, and a flexible stem extending proximally from the distal end, flexible with respect to the at least one barb and distal tip. The flexible stem may be configured to flex in cooperation with a force exerted on the anchor. The at least one anchor may be configured to engage with the tissue and resist proximal movement.

The driver may be configured to rotate about the applicator shaft, and further may be configured to index the driving of anchors in line with each spring arm.

The applicator may be removable from the prosthetic valve by exertion of a distal force on the applicator shaft to release the spring arms from engagement with the pliable ring, and exertion of a proximal force on the applicator shaft to draw the applicator proximally through the prosthetic valve.

The firing arm may be hinged to the driver at a proximal end of the firing arm, or may be hinged to the driver at a distal end of the firing arm.

In accordance with example embodiments of the present invention, a surgical device includes a driver having a distal end, at least one firing arm in hinged communication with the distal end of the driver, each including at least one anchor outlet, having a refracted position parallel to the driver, and a firing position in which the firing arm is directed proximally and radially outward, a guide situated within the driver, and a firing mechanism connected to the guide and configured to transfer force from the guide in the proximal and radially outward direction of the firing arm in the firing position.

In accordance with example embodiments of the present invention, a surgical device includes a pliable ring collapsible for insertion into a catheter and expandable upon ejection from the catheter, an applicator having one or more spring arms configured to exert a force on the ring to ring the ring into apposition with tissue, and a driver having at least one firing arm configured to drive at least one anchor into the ring and the tissue to affix the ring to the tissue. The spring arms may further be configured to conform the ring to the contours of the surrounding tissue. The spring arm may exert the force on the ring while the driver drives the at least one anchor into the ring and the tissue to affix the ring to the tissue.

The surgical device may include an applicator shaft passing through the ring and terminating in the one or more spring arms connecting the distal end of the applicator shaft to the ring, such that a proximal force applied to the applicator shaft is transferred to the one or more spring arms and to the pliable ring, and the firing arm may be in hinged communication with the driver, having a retracted position in which the firing arm is in parallel with the applicator shaft, and a firing position in which the firing arm is directed toward the ring.

The driver may include a plurality of firing arms, and the plurality of firing arms may fire a plurality of anchors simultaneously.

The firing position may be perpendicular to the applicator shaft. The firing position may also be less than 90 degrees from the retracted position.

The firing arm may be hinged to the driver at a proximal end of the firing arm, or may be hinged to the driver at a distal end of the firing arm.

Further features and aspects of example embodiments of the present invention are described in more detail below with reference to the appended Figures.

### BRIEF DESCRIPTION OF THE DRAWINGS

FIG. 1 is an illustration of the heart valve replacement prosthetic, the applicator shaft, the spring arms, and the driver, in accordance with an example embodiment of the present invention.

FIG. 2 is an illustration of the heart valve replacement prosthetic, the applicator shaft, and the spring arms, in accordance with an example embodiment of the present invention.

FIG. 3 is an illustration of the heart valve replacement prosthetic, the applicator shaft, the spring arms, and the driver, in accordance with an example embodiment of the present invention.

FIG. 4 is an illustration of the heart valve replacement prosthetic, the applicator shaft, the spring arms, and the driver, in accordance with an example embodiment of the present invention.

FIG. 5 is an illustration of the heart valve replacement prosthetic, the applicator shaft, the spring arms, and the driver, in accordance with an example embodiment of the present invention.

FIG. 6 is an illustration of the heart valve replacement prosthetic, the applicator shaft, the spring arms, and the driver, in accordance with an example embodiment of the present invention.

FIG. 7 is an illustration of the heart valve replacement prosthetic, the applicator shaft, the spring arms, and the driver, in accordance with an example embodiment of the present invention.

FIG. 8 is an illustration of the heart valve replacement prosthetic, the applicator shaft, the spring arms, and the driver, in accordance with an example embodiment of the present invention.

FIG. 9 is an illustration of the heart valve replacement prosthetic in accordance with an example embodiment of the present invention.

FIG. 10 is an illustration of the heart valve replacement prosthetic in accordance with an example embodiment of the present invention.

FIG. 11 is an illustration of an anchor in accordance with an exemplary embodiment of the present invention.

FIG. 12 is an illustration of an anchor in accordance with an exemplary embodiment of the present invention.

FIG. 13 is an illustration of the driver of the heart valve replacement prosthetic in accordance with an example embodiment of the present invention.

FIG. 14 is an illustration of a cross-sectional view of the driver of the heart valve replacement prosthetic in accordance with an example embodiment of the present invention.

FIG. 15 is an illustration of the driver of the heart valve replacement prosthetic in accordance with an example embodiment of the present invention.

FIG. 16 is an illustration of a cross-sectional view of the driver of the heart valve replacement prosthetic in accordance with an example embodiment of the present invention.

FIG. 17 is an illustration of the driver of the heart valve replacement prosthetic in accordance with an example embodiment of the present invention.

FIG. 18 is an illustration of a cross-sectional view of the driver of the heart valve replacement prosthetic in accordance with an example embodiment of the present invention.

FIG. 19 is an illustration of the driver of the heart valve replacement prosthetic in accordance with an example embodiment of the present invention.

FIG. 20 is an illustration of a cross-sectional view of the driver of the heart valve replacement prosthetic in accordance with an example embodiment of the present invention.

### DETAILED DESCRIPTION

As set forth in greater detail below, example embodiments of the present invention allow for the reliable and effective delivery, implantation, and fixation of a heart valve replacement prosthetic, such that the prosthetic can effectively address heart valve failure.

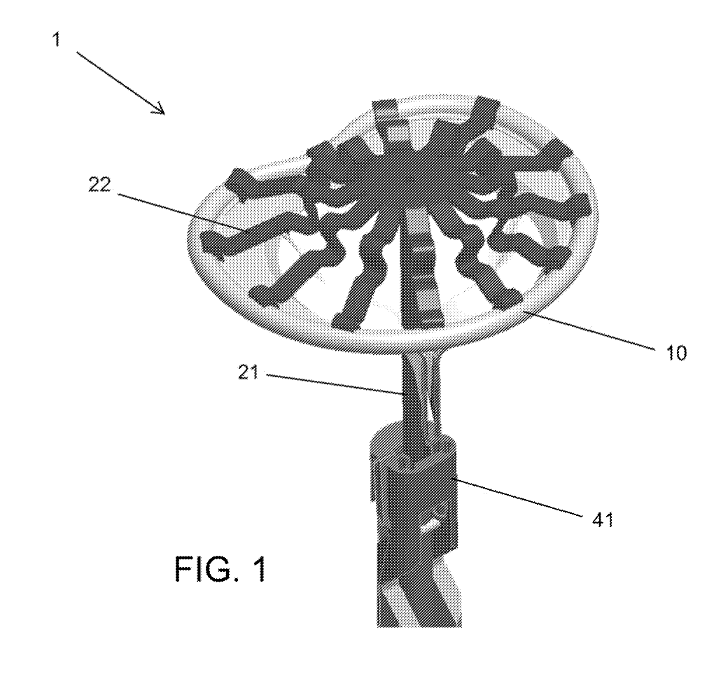

An exemplary embodiment of the present invention is present in FIG. 1. FIG. 1 illustrates heart valve replacement prosthetic 1 having ring 10, which, in an exemplary embodiment, may be elastic. FIG. 1 further illustrates applicator 20 having applicator shaft 21 and spring arms 22. Spring arms 22 may be affixed to the distal end of the applicator shaft 21, and may connect the distal end of the applicator shaft 21 to the ring 10 of the replacement prosthetic 1. FIG. 1 further illustrates driver 40, as will be described in more detail below.

As will be generally understood, as described by, for example, U.S. Pat. No. 7,621,948, the entirety of which is hereby incorporated by reference as if fully disclosed herein, the replacement prosthetic 1 of the present invention may be delivered to an implant site by first collapsing the replacement prosthetic 1 into a collapsed or folded position, such that the prosthetic fits within a cavity of a catheter. The catheter, including the collapsed or folded prosthetic, is advanced percutaneously to an implant site. Once the distal end of the catheter is adjacent to the implant site, the collapsed prosthetic may be pushed or forced through the distal end of the catheter.

Heart valve replacement prosthetic 1 may be formed of compliant, elastic material such as deformable plastic or nitinol, such that once the collapsed prosthetic emerges from the distal end of the catheter, the ring 10 may elastically return to an uncollapsed, or expanded formation, as illustrated in FIGS. 1 to 10. The prosthetic 1, including ring 10, may be maneuvered in the implant site, where it may be pressed into position in the implant site.

As further illustrated in FIGS. 1 to 10, ring 10 may, in an exemplary embodiment of the present invention, take on the shape of the implant site, which may be an irregular shape (e.g., non-circular). In the exemplary embodiments illustrated in FIGS. 1 to 10, the ring 10 is formed to a non-circular, irregular shape. In this manner, the heart valve replacement prosthetic of the present invention may be adapted to a wide variety of implant sites, to address a wide variety of heart valve failures.

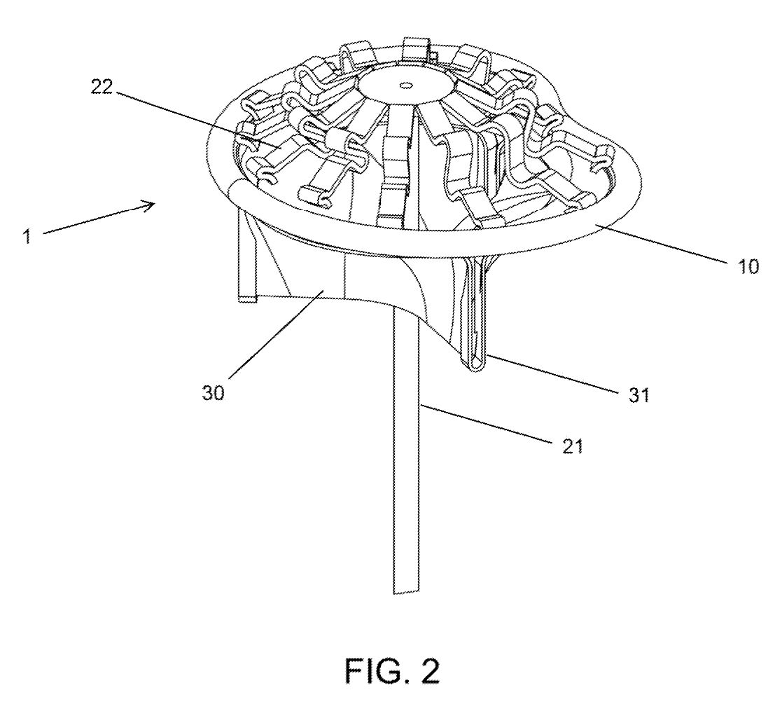

As illustrated in FIG. 2, heart valve replacement prosthetic 1 further includes leaflets 30, which perform the valve function. Leaflets 30 are connected to ring 10, and are further held in proper position by valve struts 31. Leaflets 30 are configured to prevent the flow of fluid in a first fluid flow direction, and to permit the flow of fluid in a second fluid flow direction.

FIGS. 2 through 8 illustrate an exemplary embodiment of the fixing of the replacement prosthetic 1 to the tissue of the implant site.

As illustrated in FIG. 2, replacement prosthetic 1 is expanded from the catheter, with ring 10 in a nearly fully expanded formation. Applicator shaft 21 extends from the proximal direction through and to the distal side of the ring 10. Spring arms 22 are connected to the distal end of the applicator shaft 21, such that the distal end of the applicator shaft 21 forms the apex of a conical shape formed by the spring arms 22 about the axis of the applicator shaft 21. Once delivered to the implant site, applicator shaft 21 may be used to press ring 10 into the tissue of the implant site, by pulling the applicator shaft 21 in a proximal direction, such that the force in the proximal direction is transferred to the spring arms 22, which in turn exert a force in a proximal and radial direction against the ring 10. Because the spring arms include spring elements 23, such as springs or spring-like ribbons, each spring arm is flexible to absorb force independently of the other spring arms. In this manner, ring 10 is further able to achieve an irregular shape, to meet the shape of any implant tissue. FIGS. 2 to 8 illustrate various spring arms 22 being extended or compressed to a different degree.

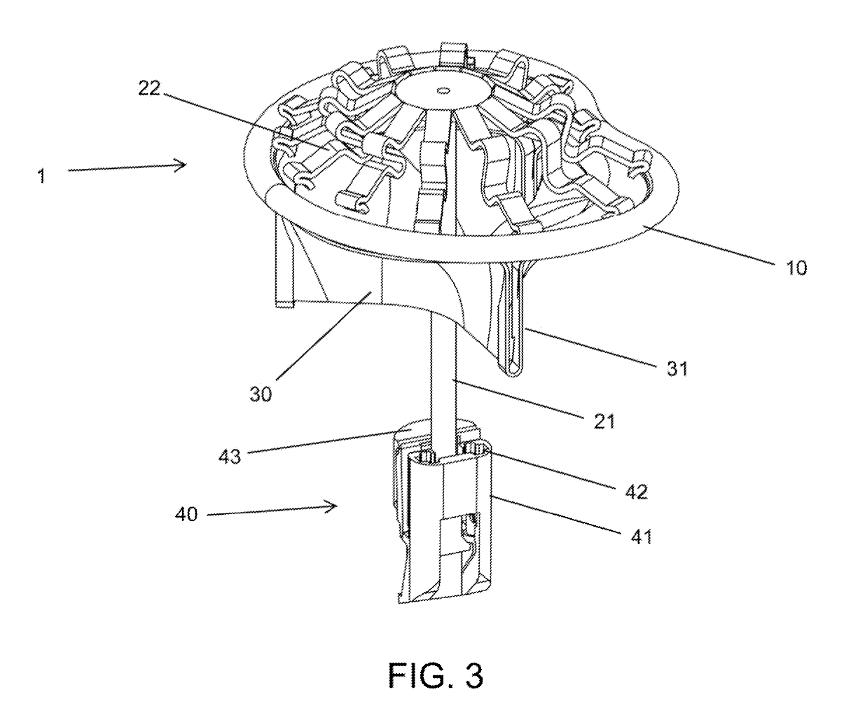

As illustrated in FIG. 3, once the prosthetic 1 is in place at the implant site, driver 40 may be actuated to fasten the ring 10 to the tissue of the implant site. Driver 40 includes firing arm 41, having anchor outlets 42, and a guide 43. Driver 40 may be operated to slide or otherwise move along the applicator shaft 21. Guide 43 and firing arm 41 may be situated on opposite sides of the applicator shaft 21, as illustrated in FIG. 3.

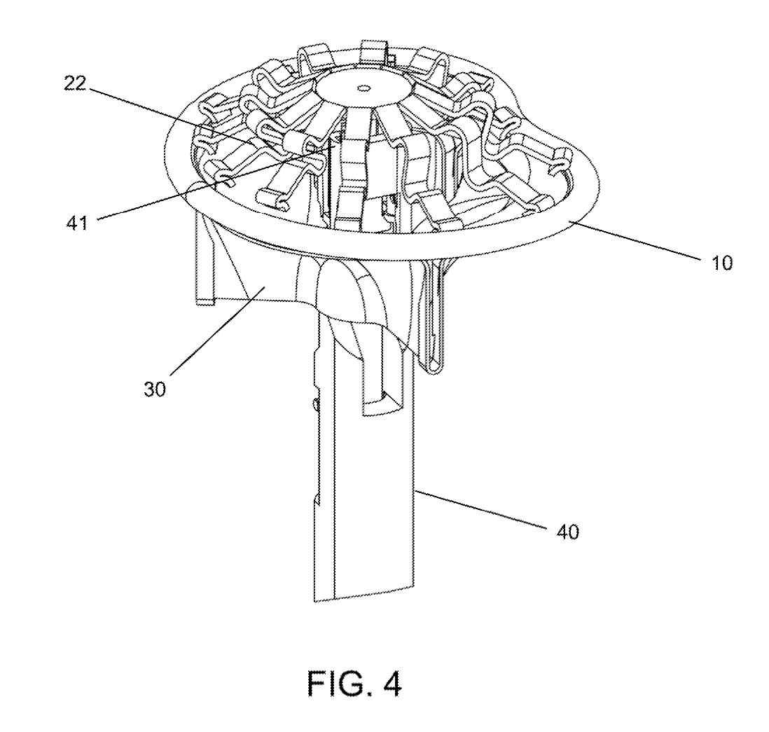

As illustrated in FIG. 4, driver 40 is moved to the distal end of the applicator shaft 21, where the applicator shaft 21 meets the spring arms 22.

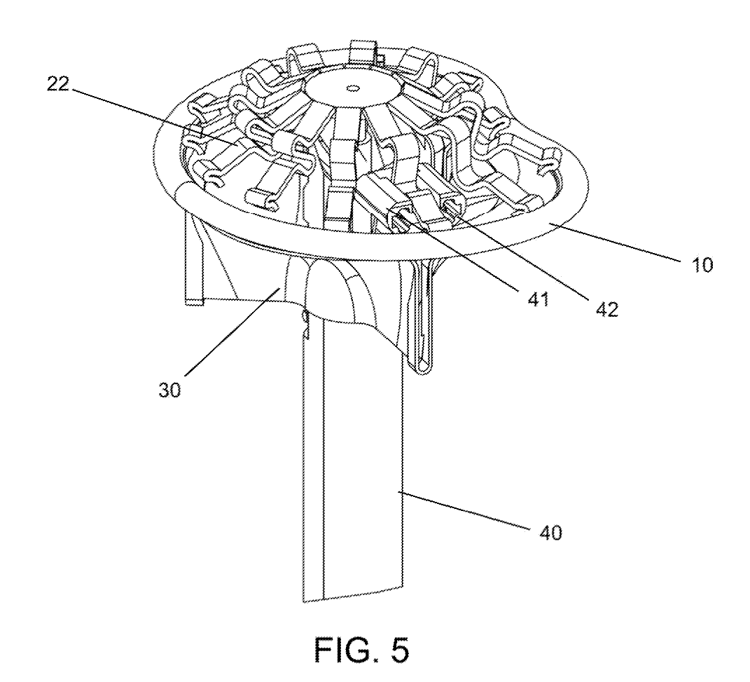

As illustrated in FIG. 5, firing arm 41 is configured to rotate from a position aligned with the axis defined by the applicator shaft 21 to a position directed radially away from the applicator shaft 21, so that the anchor outlets 42 of the firing arm are directed towards the ring 10.

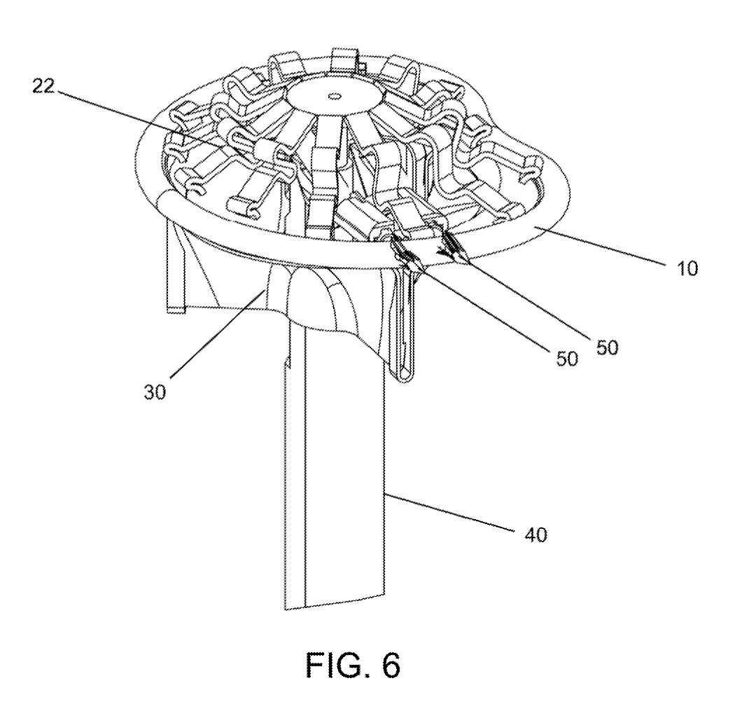

As illustrated in FIG. 6, firing arm 41 may be configured to drive anchors 50 through anchor outlets 42. Anchors 50 may be driven through ring 10, and into surrounding tissue of the implant site, fixing or fastening the ring 10 to the tissue of the implant site.

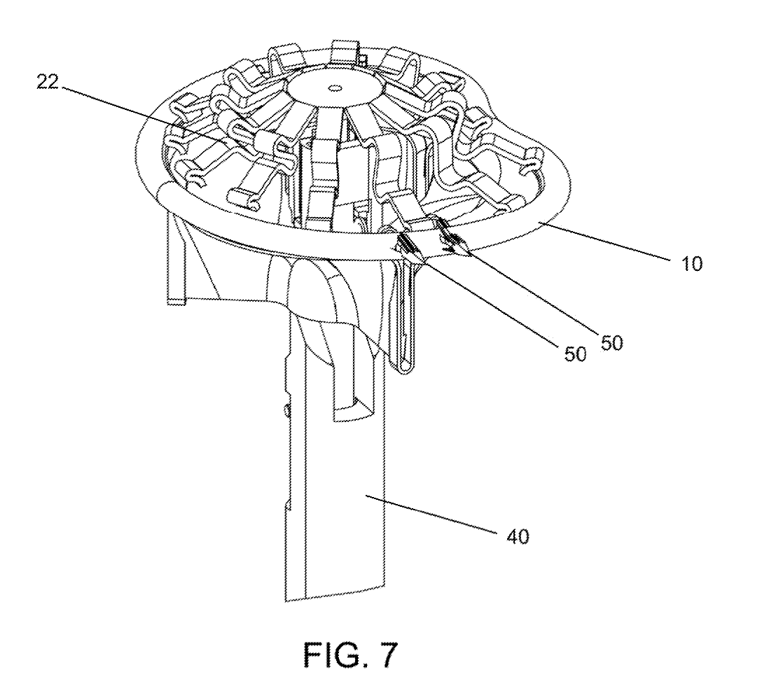

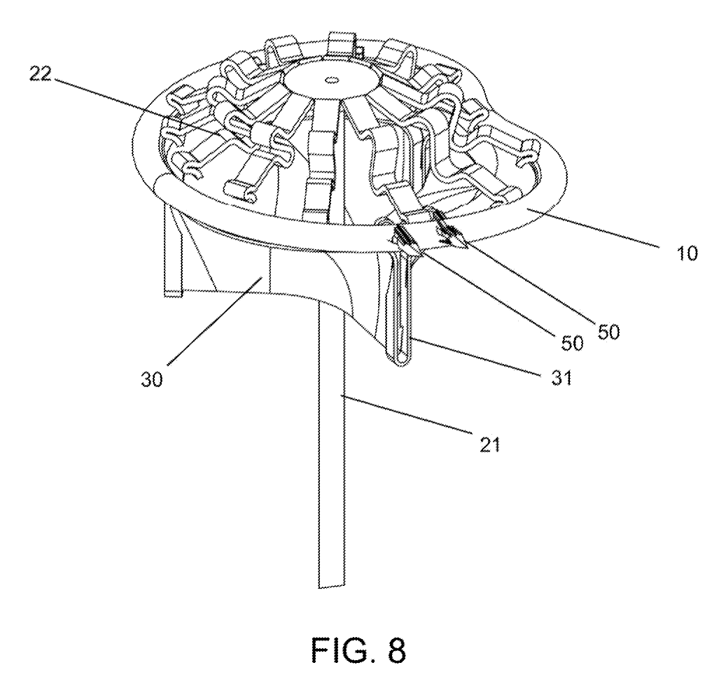

As illustrated in FIG. 7, once anchors 50 are driven into ring 10 and the surrounding tissue, firing arm 41 may be rotated back into alignment with the axis defined by the applicator shaft 21, so that the driver 40 may be retracted from the distal end of the applicator 20, as illustrated in FIG. 8.

In an exemplary embodiment of the present invention, the driving of anchors 50 may be repeated by driver 40 and firing arm 41, so as to drive anchors 50 around the ring 10. A plurality of anchors 50 may be loaded into a cartridge or tray of anchors, such that additional anchors may be loaded into a position to be driven into ring 10 and the surrounding tissue. Driver 40 may index the driving of each anchor 50 to the position of each spring arm 22 about the periphery of the ring 10. In the alternative, applicator shaft 21 may have grooves or other markings to which driver 40 may index the driving of each anchor 50.

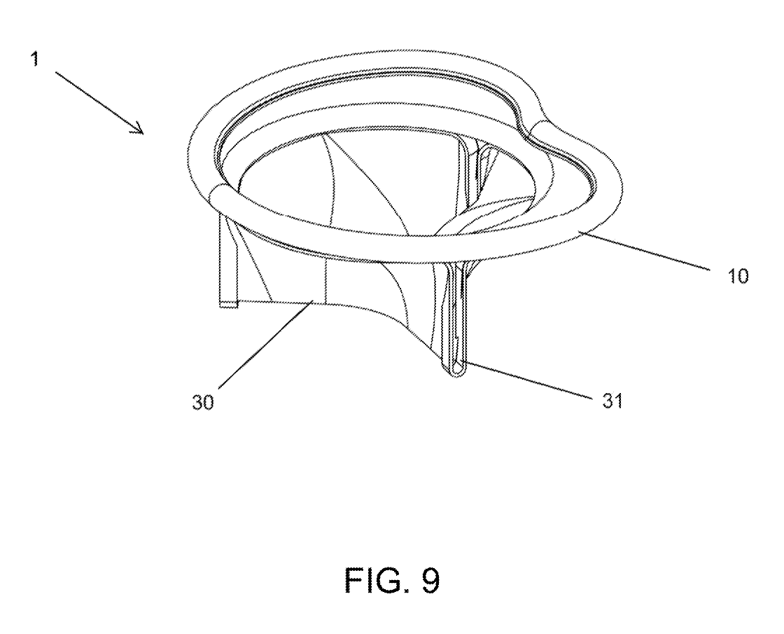

As illustrated in FIG. 9, heart valve replacement prosthetic 1 is shown alone, absent applicator 20, driver 40, or anchors 50.

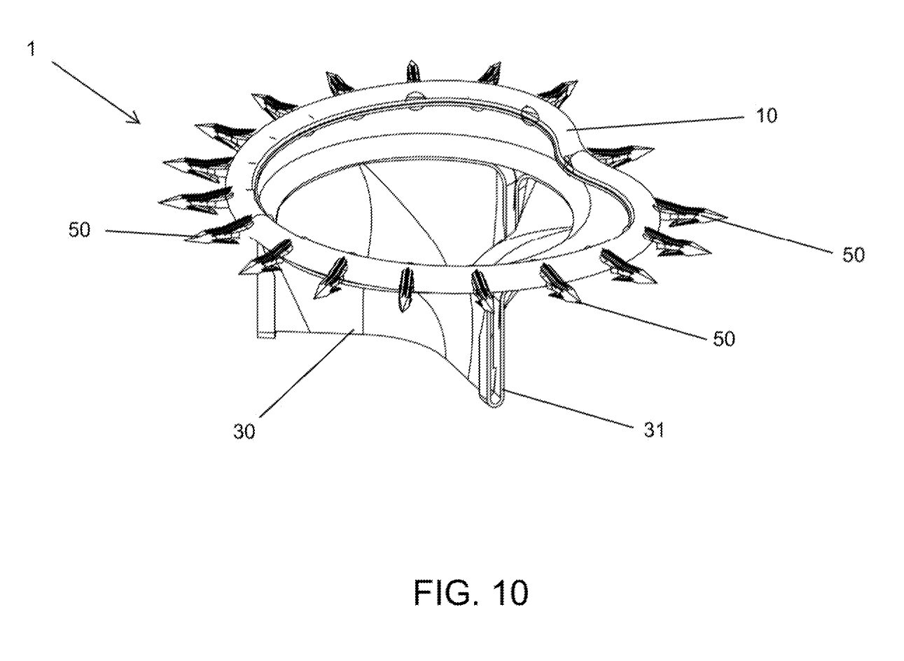

FIG. 10 illustrates the heart valve replacement prosthetic 1 after the driver 40 has completed driving a plurality of anchors 50 about the periphery of ring 10, and further after the applicator 20, including applicator shaft 21 and spring arms 22, have been withdrawn. To withdraw the applicator 20, the applicator shaft 21 may be moved in a distal direction, extending spring arms 22 further beyond the distal side of the ring 10, and permitting the conical structure formed by the spring arms 22 to collapse. Once collapsed, the spring arms 22 may be permitted to pass through ring 10 with the withdrawal of the applicator shaft 21, so that the entire applicator 20 may be withdrawn from the implant site.

Anchors 50 may be any of the anchors described in U.S. Patent Provisional Application No. 61/296,868, filed Jan. 20, 2010, U.S. patent application Ser. No. 13/010,766, filed Jan. 20, 2011, U.S. patent application Ser. No. 13/828,256, filed Mar. 14, 2013, U.S. patent application Ser. No. 13/843,930, filed Mar. 15, 2013, and U.S. patent application Ser. No. 14/301,106, filed Jun. 10, 2014, each of which is incorporated by reference in their entirety as if fully disclosed herein.

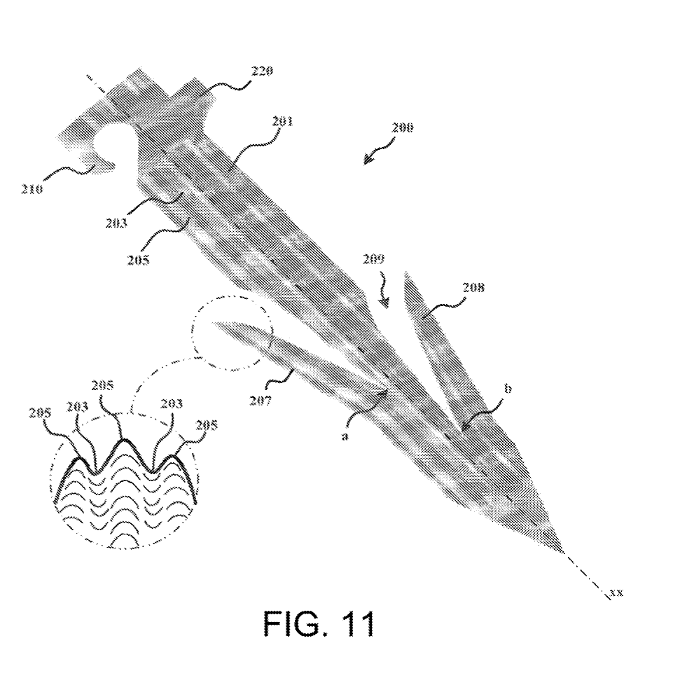

For example, FIG. 11 shows an anchor or implant 200 which is configured to be driven into a tissue. The anchor 200 includes a corrugated body 201. The body 201 includes grooves 203 that extend axially along the length of the body 201. Thus, extending circumferentially around the body 201, a plurality of grooves 203 alternate with a plurality of ridges 205. Further, the anchor body 201 includes a pair of wings or split portions 207 and 208. The split portions 207 and 208 are formed by respective splits or cuts 209 into the body 201. In this regard, the splits 209 may be formed by making a cut radially into the body 201 and extending in an axial direction. Thus, the two split portions 207 and 208 are attached to the remainder of the body 201 at a distal position and extend proximally to free ends. The free ends include a plurality of sharp protrusions along a curved surface. These points are formed due to the corrugations. In particular, the ridges 205 form the sharp protrusions, as illustrated in the inset partial side view in FIG. 11, which are advantageous for gripping tissue and preventing distal sliding of the anchor 200. Although each split portion 207 and 208 includes three such protrusions as illustrated, it should be understood that the anchor 200 may be designed such that one or more of the split portions has any other number of protrusions, including a single sharp protrusion. For example, if a larger number of sharp protrusions are desired, the body 201 could be more densely corrugated (i.e., a greater number of alternating grooves 203 and ridges 205 could be provided) and/or the angle of the cut or slice could be adjusted. Further, the length of proximal extension of the projections may be adjusted by varying the depth of the grooves 203 with respect to the ridges 205.

The split portions 207 and 208 do not substantially impede distal insertion into tissue but resist proximal movement from an insertion location by engaging the tissue. It has been discovered that the combination of the pointed and/or sharp-edged proximal ends of the split portions 207 and 208 with the alternating ridges on the proximal end of the split portions creates improved performance.

Further, the split portions or wings 207 and 208 are axially offset from each other. For example, split 207 is axially located at position along axis xx and split 208 is axially located at position b along axis xx. This allows for greater structural strength of the other portions of the body 201 as compared to a non-offset configuration. In particular, since the cuts progress continually radially inward as they progress distally, a non-offset portion would have a substantially smaller amount of material in cross-section at the distal end of the cut. This would lead to a mechanically weak point or region along the axis of the body and could lead to mechanical failure, especially in anchors of small dimensions. Although the anchors 200 utilize a pair of wings 207 and 208 to anchor the anchors 200 against proximal retraction from a tissue, it should be appreciated that any number of wings may be provided, and that as an alternative or in addition to the wings 207 and 208, any other appropriate anchoring structure(s), e.g., anchoring filaments, may be provided.

The distal tip of the anchor 200 is pyramidal, with a sharp point, and a plurality of surfaces separated by edges that converge at the sharp point. Although four planar surfaces are provided, it should be appreciated that any appropriate suitable number of surfaces may be provided and that one or more or all of the surfaces may be non-planar.

The anchor 200 may include one or more shoulders, formed by the junction of a wing 207, 208, with the body 201, or otherwise defined by the area of the anchor 200 where the wing 207, 208, extends proximally and radially outwardly from the distal end, or distal thereto. As illustrated in FIG. 11, wings 207, 208, have a relaxed, uncompressed position, but may be compressed to a second, compressed position, in closer approximation with the body 201. Further, the body 201 may be flexible, such that forces experienced in the proximal end may influence the position of the body or stem 201 with respect to the wings 207, 208, and the distal end.

The anchor 200 may be produced by first forming the body 201 with the corrugations, e.g., by injection molding or extrusion, and subsequently forming split portions 207 and 208, e.g., by cutting radially into the side of the body 201. As illustrated, the cut is curved, with an angle (at the proximal entry point), relative to the longitudinal axis xx of the body 201, that gradually decreases from the proximal initial cutting location toward the distal end of the anchor 200 and eventually becoming linear. Although the split or cut of the illustrated example is made with a curved or varying angle with respect to the longitudinal axis xx of the body 201, it should be understood that any appropriate cut, including a linear cut, may be made.

Although the anchor 200 includes two wings or split portions spaced equally around the radial periphery of the body 201, it should be appreciated that any number of split portions, including a single split portion may be provided and at any appropriate spacing around the radial periphery of the anchor 200.

Modern manufacturing processes allow for near nano technology applications. This allows the anchors to be manufactured in a size and complexity that may not have been possible in years past. The anchor 200 may be injection molded of either absorbable or non-absorbable polymers and then processed (e.g., by cutting) to add the features of the wings 207 and 208. Although the anchors 200 are formed of polymer, it should be appreciated that any appropriate material may be used, e.g., metal or a composite material. The anchors 200 may have a diameter of, e.g., one millimeter, or approximately one millimeter, and a length that is in a range from, e.g., 5 millimeters to 10 millimeters. According to some example embodiments, the diameter is less than one millimeter. According to some example embodiments, the diameter is in a range from 0.8 millimeters to 1.2 millimeters. It should be understood, however, that other dimensions may be provided.

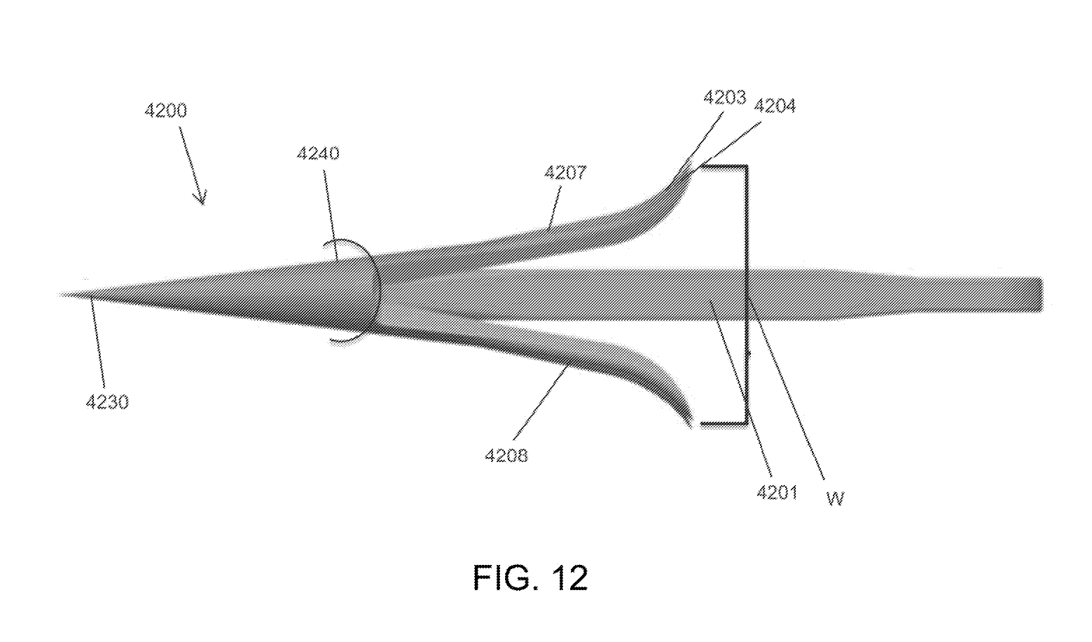

In an exemplary embodiment of the present invention, the anchor 4200 illustrated in FIG. 12 is described. Anchor 4200 includes a distal tip 4230, and a stem 4201 extending proximally from the base of distal tip 4230. Stem 4201 joins the base of distal tip 4230 at shoulder 4240. Wings or barbs 4207, 4208 extend proximally, and, to some degree, radially, from the base of distal tip 4230, and join the base of distal tip 4230 at shoulder 4240. Barbs 4207, 4208 extend proximally and radially from the distal tip 4230 to free ends. The free ends may flare further radially outward, as illustrated in FIG. 12. Unlike the wings or split portions 207, 208 described above, wings or barbs 4207, 4208 are not formed from cuts or splits to the body of the anchor, so that the thickness of stem 4201 may be unaffected by the inclusion of barbs 4207, 4208. Wings or barbs 4207, 4208 may have a relaxed, uncompressed position, illustrated in FIG. 12. In the uncompressed position, barbs 4207, 4208 are unbiased, having a barb opening W. Barbs 4207, 4208 may be compressed into closer approximation with stem 4201. Varying amounts of compression may be applied to the barbs, such that the greater the compression, the closer approximation of the barbs to the stem. Barbs 4207, 4208 may include protrusions at the free ends of the barbs, to engage with tissue once the anchor has been deployed. While two barbs 4207, 4208 are illustrated, it should be appreciated that any number of barbs may be provided. Similarly, any number of protrusions at the free ends of the barbs may be provided, including one sharp protrusion.

Stem 4201 may be flexible, able to be bent or flexed with respect to barbs 4207, 4208 and distal tip 4230. Once deployed into tissue, a flexible stem provides for a different profile of forces acting on the anchor 4200, as compared to an anchor having a rigid or stiff stem. A flexible shaft, able to flex in relation to the barbs and the distal tip, creates a living hinge between these elements of the anchor. Forces acting on the anchor from its proximal end may be at least partially absorbed by the flexible stem, so that the impact of these forces on the wings or barbs of the anchor may be reduced. In certain tissue environments, a flexible shaft may be more likely to prevent a levering action by the anchor, and may thereby prevent the anchor from partially or even completely pulling out of the tissue.

Further, the anchors 50, 200, 4200 may include any of the features of the fasteners or other analogous implants disclosed in U.S. Provisional Patent Application Ser. No. 61/296,868, filed on Jan. 20, 2010, in U.S. patent application Ser. No. 13/010,766, filed on Jan. 20, 2011, and U.S. patent application Ser. No. 14/301,106, filed on Jun. 10, 2014, each of which is incorporated by reference in its entirety as if fully disclosed herein, and may be driven using any mechanism disclosed therein.

To fire the anchors, a force delivery system may be situated at the proximal end of the driver. The force delivery system may use any mechanisms of nearly instantaneous force transfer, such as springs, gas, compressed fluid, or the like. Force is transferred through the shaft of the driver, which may be a rigid shaft or a flexible shaft, depending on the application. The force is used to displace a firing mechanism at the distal end of the shaft, which in turn exerts a driving force on the anchors to drive the anchors from the firing arms and into the prosthetic valve and the surrounding tissue. The driving force may result from a pushing force delivery system, which directs force in the distal direction of the driver, or a pulling force delivery system, which directs force in the proximal direction of the driver, depending on the application.

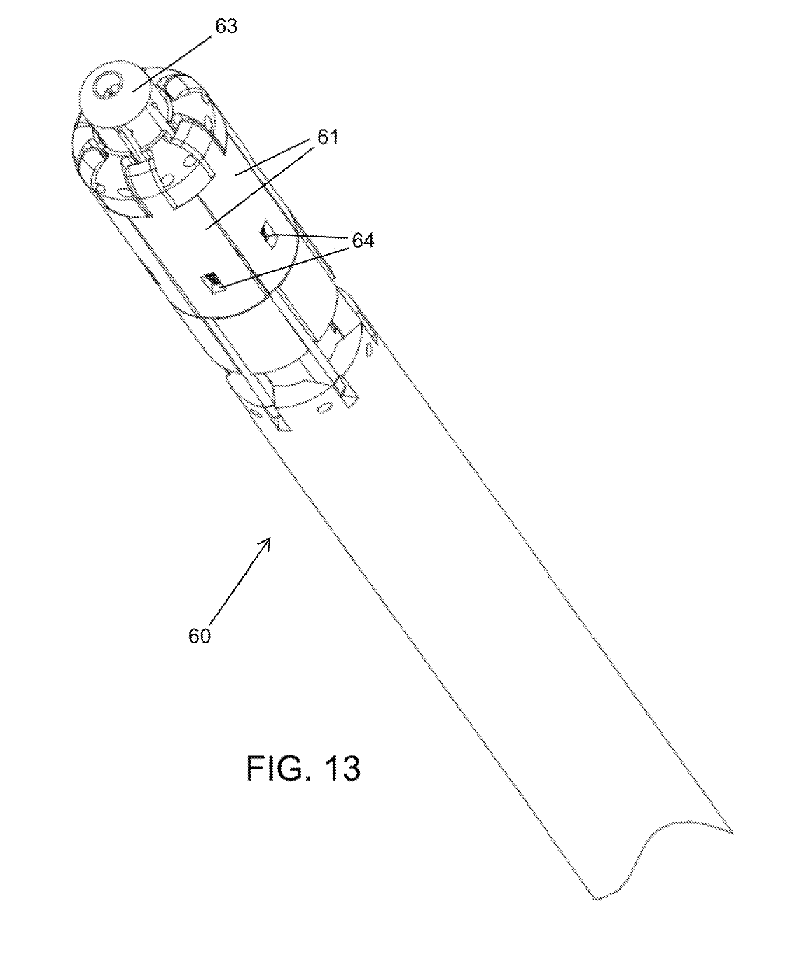

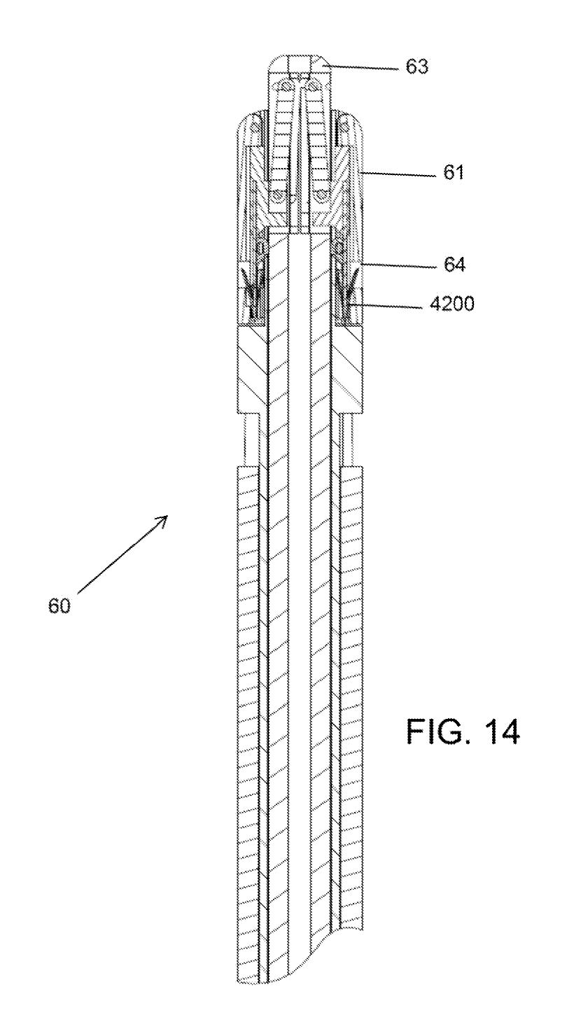

In an exemplary embodiment of the present invention, a plurality of firing arms may be provided around the applicator, as illustrated in FIGS. 13 to 20. FIGS. 13 and 14 show driver 60 having, at its distal end, a plurality of firing arms 61 situated annularly around tubular guide 63. Guide 63 may surround an applicator shaft, much like guide 43. Firing arms 61 are in a refracted position against the driver and parallel to the axis of the driver. FIG. 14 also shows anchors 4200 provided in the firing arms 61. Windows 64 allow for the barbs 4207, 4208 to be stored in the firing arm 61 in their relaxed position before being driven from the anchor outlet and into the ring 10 and the surrounding tissue.

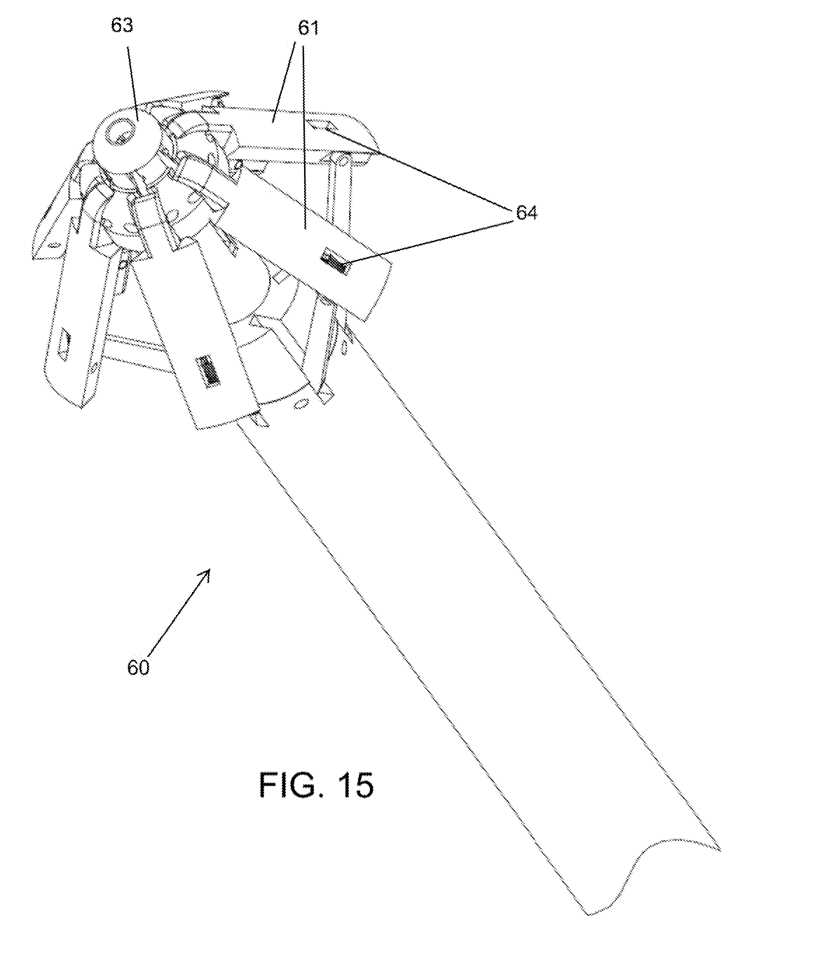

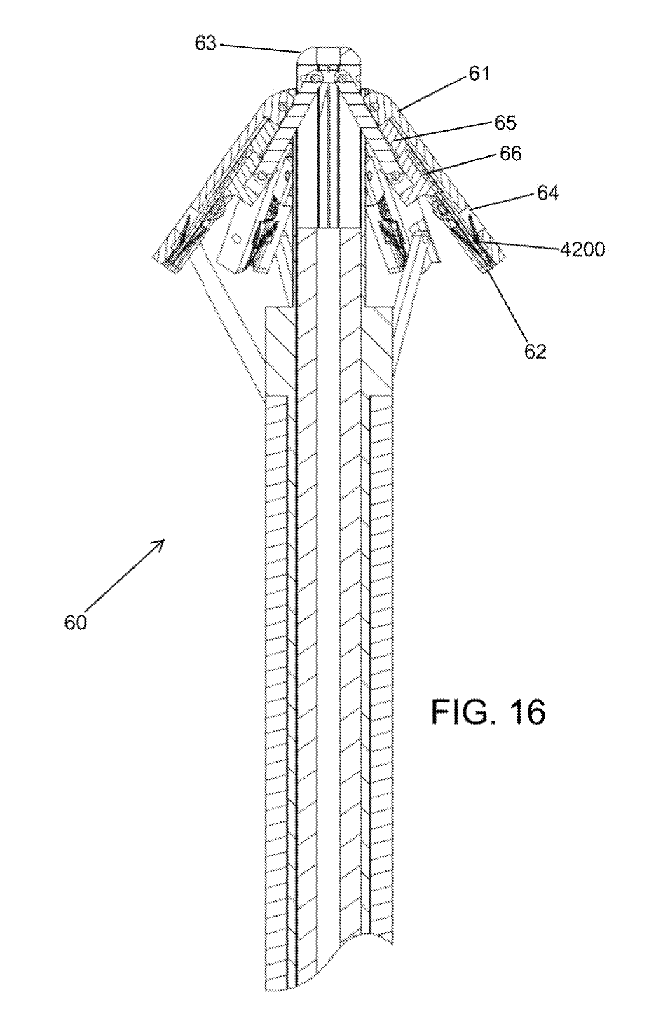

FIGS. 15 and 16 illustrate the driver 60 in which the firing arms have been moved from the retracted position to a firing position. The movement of the firing arms from the retracted position of FIGS. 13 and 14 to the firing position of FIGS. 15 and 16 may be achieved by manual or electric actuation of a translating force, for example, by a screw or a sliding mechanism, or by any other mechanical operation. Firing arms 61 are hinged to driver 60 at the distal end of the driver, so that the firing arms 61 open radially outwardly from a position proximal to the hinges and the distal end of the driver 60. The firing position of the firing arms may be at an angle of less than 90 degrees from the axis of driver 60. An acute angle of firing arms 61 allows for an angled anchor delivery into ring 10 and the surrounding tissue, and therefore greater control of the placement of the anchors in the surrounding tissue.

As illustrated in FIG. 16, firing arms 61 include firing mechanisms 65 and fingers 66, for driving anchors 4200 through anchor outlets 62. Guide 63 is connected to firing mechanisms 65 and fingers 66, so that the application of a proximal or pull force to the guide will translate the force in a proximal direction to the firing mechanisms 65 and finger 66. Fingers 66 abut shoulder 4240 of anchor 4200, and may transfer the pull force from the guide 63 and firing mechanism 65 to anchor 4200.

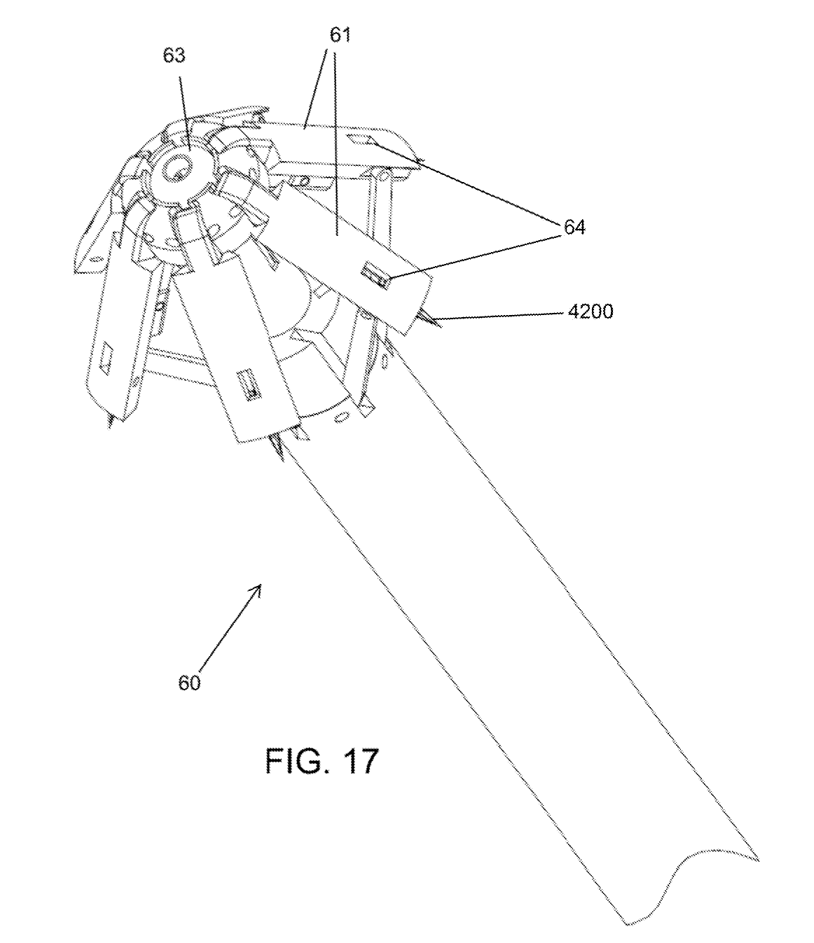

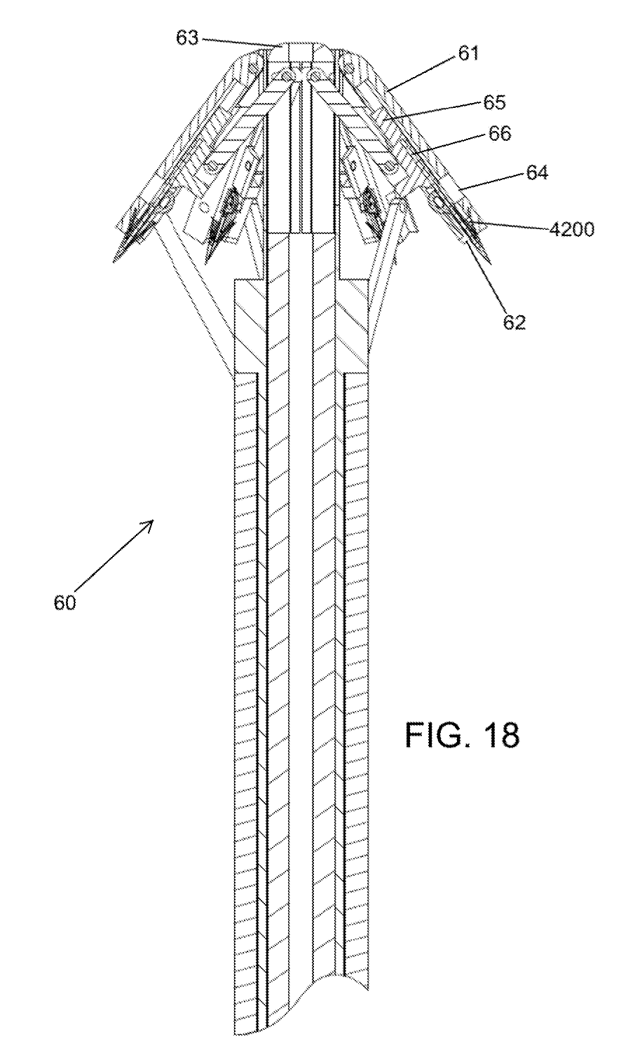

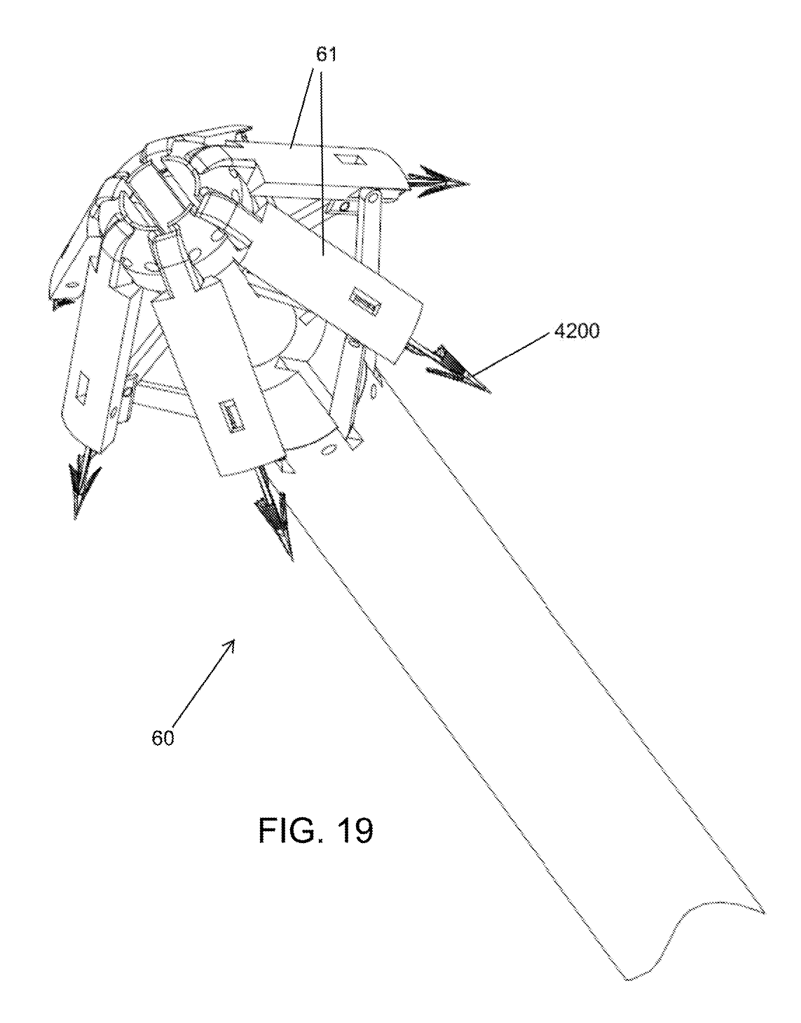

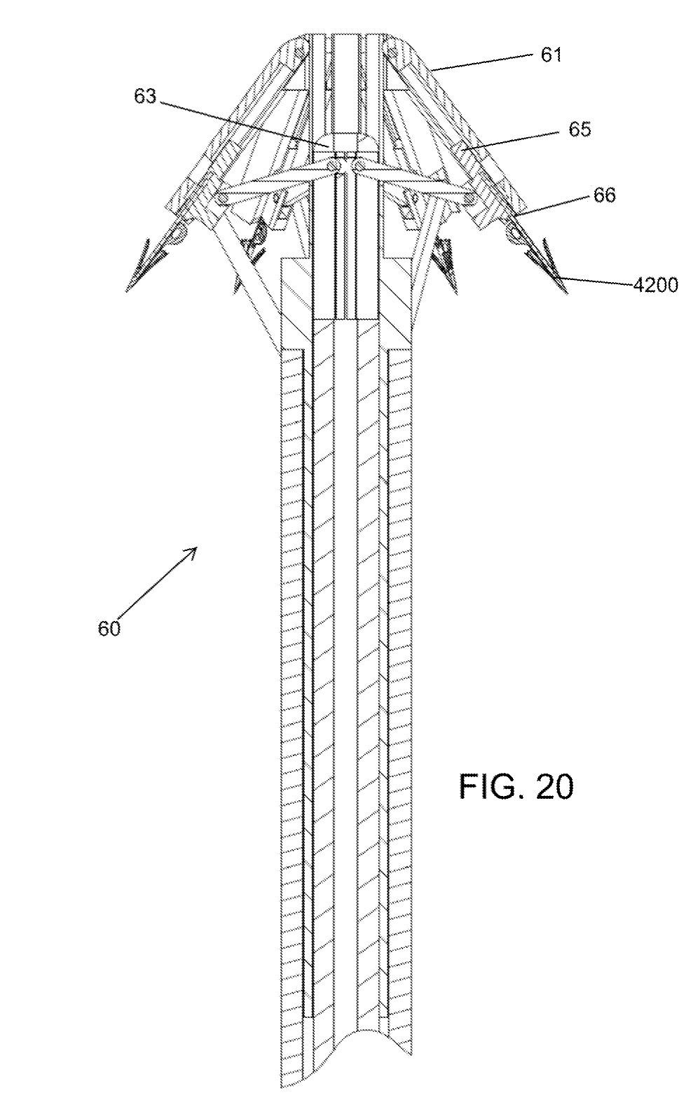

FIGS. 17, 18, 19, and 20 illustrate the firing of anchors 4200. Anchors 4200 are fired by exertion of a proximal or pulling force, pulling guide 63 in a proximal direction with respect to driver 60. As shown in FIGS. 17 and 18, guide 63 is drawn nearly level with the hinged ends of firing arms 61, and, as shown in FIGS. 19 and 20, guide 63 is drawn to a recessed position with respect to the hinged ends of firing arms 61. The proximal force drawing guide 63 is transferred to firing mechanisms 65, and in turn to finger 66, which then transfers the driving force to shoulder 4240 of anchor 4200, driving anchor 4200 through anchor outlet 62, into ring 10 and the surrounding tissue. In this manner, all of the firing arms 61 may fire anchors 4200 at the same time.

Further, any of the implantable elements described herein, e.g., anchors 50, 200, 4200, and ring 10, leaflets 30, valve struts 31, or any other element of heart valve replacement prosthetic 1, may be formed wholly or partly of a material absorbable into the patient's body, or of a non-absorbable material, depending on, e.g., the specific application. For example, these elements may be formed of polyglycolic acid (PGA), or a PGA copolymer. These elements may also, or alternatively, be formed of copolymers of polyester and/or nylon and/or other polymer(s). Moreover, these elements may contain one or more shape-memory alloys, e.g., nitinol, spring-loaded steel or other alloy or material with appropriate properties.

Absorbable materials may be advantageous where there is a potential for misfiring or improper locating of the various implants. For example, in a situation where the driver drives an anchor 50, 200, 4200 at an unintended location, or where the tissue does not properly receive the anchor 50, 200, 4200, the anchor 50, 200, 4200, even where not needed, would be relatively harmless, as it would eventually absorb into the patient's body.

Although particular example heart valve replacement prosthetic systems have been described above, the systems and devices described here are in no way limited to these examples.

Although the present invention has been described with reference to particular examples and exemplary embodiments, it should be understood that the foregoing description is in no manner limiting. Moreover, the features described herein may be used in any combination.

What is claimed is:

1. A surgical device, comprising:
a prosthetic valve device having a pliable ring;
an applicator having an applicator shaft passing through the ring and terminating at a distal end having one or more spring arms connecting the distal end of the applicator shaft to the ring, such that a proximal force applied to the applicator shaft is transferred to the one or more spring arms and to the pliable ring; and
a driver having a guide situated annularly about the applicator shaft, and at least one firing arm including at least one anchor outlet, the driver configured to slide between a proximal position and a distal position along the applicator shaft;
wherein the at least one firing arm is in hinged communication with the driver, such that in the proximal position of the driver the at least one anchor outlet is situated in parallel with the applicator shaft, and in the distal position of the driver the at least one anchor outlet is directed toward the pliable ring; and
wherein the driver is configured to exert a driving force on at least one anchor to drive the anchor into the pliable ring.

2. The surgical device of claim 1, wherein the prosthetic valve is delivered to an implant site by a catheter, the pliable ring being folded for insertion into the catheter and expandable once pushed from a distal end of the catheter.

3. The surgical device of claim 1, wherein the prosthetic valve is implanted at an implant site, and the driver is configured to drive the anchor at least partially through the ring and into tissue surrounding the implant site.

4. The surgical device of claim 1, the at least one anchor comprising:
a distal end tapered to a distal tip configured to pierce tissue;
at least one barb extending proximally and radially outwardly from the distal end to a free end, including a radially exterior surface and a radially interior surface; and
a flexible stem extending proximally from the distal end, flexible with respect to the at least one barb and distal tip.

5. The surgical device of claim 5, wherein the flexible stem is configured to flex in cooperation with a force exerted on the anchor.

6. The surgical device of claim 1, wherein the at least one anchor is configured to engage with the tissue and resist proximal movement.

7. The surgical device of claim 1, wherein the driver is configure to rotate about the applicator shaft.

8. The surgical device of claim 7, wherein the driver is configured to index the driving of anchors in line with each spring arm.

9. The surgical device of claim 1, the applicator being removable from the prosthetic valve by exertion of a distal force on the applicator shaft to release the spring arms from engagement with the pliable ring, and exertion of a proximal force on the applicator shaft to draw the applicator proximally through the prosthetic valve.

10. The surgical device of claim 1, wherein the firing arm is hinged to the driver at a proximal end of the firing arm.

11. The surgical device of claim 1, wherein the firing arm is hinged to the driver at a distal end of the firing arm.

12. A surgical device, comprising:
a driver having a distal end,
at least one firing arm in hinged communication with the distal end of the driver, each including at least one anchor outlet, having a retracted position parallel to the driver and a firing position in which the firing arm is directed proximally and radially outward;
a guide situated within the driver; and
a firing mechanism connected to the guide and configured to transfer force from the guide in the proximal and radially outward direction of the firing arm in the firing position.

13. A surgical device, comprising:
a pliable ring collapsible for insertion into a catheter and expandable upon ejection from the catheter;
an applicator having one or more spring arms configured to exert a force on the ring so that the ring is in apposition with tissue; and
a driver having at least one firing arm configured to drive at least one anchor into the ring and the tissue to affix the ring to the tissue.

14. The surgical device of claim 13, wherein the spring arms are further configured to conform the ring to the contours of the surrounding tissue.

15. The surgical device of claim 13, wherein the spring arm exerts the force on the ring while the driver drives the at least one anchor into the ring and the tissue to affix the ring to the tissue.

16. The surgical device of claim 13, further comprising:
an applicator shaft passing through the ring and terminating in the one or more spring arms connecting the distal end of the applicator shaft to the ring, such that a proximal force applied to the applicator shaft is transferred to the one or more spring arms and to the pliable ring;
wherein the firing arm is in hinged communication with the driver, having a retracted position in which the firing arm is in parallel with the applicator shaft, and a firing position in which the firing arm is directed toward the ring.

17. The surgical device of claim 15, wherein the driver includes a plurality of firing arms, and the plurality of firing arms fire a plurality of anchors simultaneously.

18. The surgical device of claim 15, wherein the firing position is perpendicular to the applicator shaft.

18. The surgical device of claim 15, wherein the firing position is less than 90 degrees from the retracted position.

20. The surgical device of claim 15, wherein the firing arm is hinged to the driver at a proximal end of the firing arm.

21. The surgical device of claim 15, wherein the firing arm is hinged to the driver at a distal end of the firing arm.
